(19) United States
(12) Patent Application Publication
(10) Pub. No.: US 2021/0282625 A1
(43) Pub. Date: Sep. 16, 2021

(54) MOUNTING MEMBER AND ENDOSCOPIC DEVICE

(71) Applicant: Sony Olympus Medical Solutions Inc., Tokyo (JP)

(72) Inventor: Takuya TANIGUCHI, Tokyo (JP)

(73) Assignee: Sony Olympus Medical Solutions Inc., Tokyo (JP)

(21) Appl. No.: 17/149,757

(22) Filed: Jan. 15, 2021

(30) Foreign Application Priority Data
Mar. 13, 2020 (JP) 2020-044610

(51) Int. Cl. A61B 1/00; A61B 1/04; A61B 1/12

(52) U.S. Cl. CPC A61B 1/00105; A61B 1/042; A61B 18/149; A61B 1/125; A61B 1/00154; A61B 1/00128

(57) ABSTRACT

A mounting member includes: a body portion provided in a camera head including an image sensor, the body portion being rotatable about a first axis that passes through the body portion and connectable with an endoscope; a locking protrusion provided in the body portion and configured to lock with the endoscope; a detachable button configured to be movable with respect to the body portion and control attachment and detachment of the endoscope to and from the body portion; and a spring whose load applied to the locking protrusion changes according to an advancing/retreating operation of the detachable button with respect to the body portion, wherein at least one of the body portion and the detachable button includes a through-hole forming a part of a flow path of a liquid to a space in which the locking protrusion in the body portion and the spring member are arranged.

### CROSS-REFERENCE TO RELATED APPLICATION

This application claims priority from Japanese Application No. 2020-044610, filed on Mar. 13, 2020, the contents of which are incorporated by reference herein in its entirety.

### BACKGROUND

The present disclosure relates to a mounting member used in an endoscope and an endoscopic device.

In the medical field and industrial field, an endoscopic device that observes the inside of a subject such as a human and a mechanical structure is known (see, for example, JP 2000-227559 A). The endoscopic device described in JP 2000-227559 A includes an endoscope (optical telescope) that takes and emits a subject image inside a subject and endoscope camera head (TV camera) that holds the endoscope and captures the subject image emitted from the endoscope. The endoscope is held to be rotatable about an internally set optical axis with respect to the endoscope camera head. In addition, the endoscope camera head includes a mounting member and a camera head body. The mounting member has a bottomed cylindrical shape to which an eyepiece unit of the endoscope may be fitted. A locking protrusion that locks with the eyepiece unit is provided at a fitting portion of the endoscope in the mounting member. The locking protrusion is provided so as to be capable of advancing and retreating with respect to a wall surface of the mounting member according to the load applied from the eyepiece unit. The mounting member is formed with a space for arranging the above-described locking protrusion, a member that controls the advancing/retreating operation of such a locking member, and the like.

### SUMMARY

By the way, the mounting member is cleaned together with the camera head body and used repeatedly. At this time, it is necessary to clean the internal space of the mounting member, but there are cases where it is difficult for a cleaning liquid to infiltrate into the inside and it is difficult for the infiltrating cleaning liquid to be discharged.

There is a need for a mounting member and an endoscopic device capable of improving the infiltration of a cleaning liquid into the inside of the member and the discharge of the cleaning liquid to the outside.

According to one aspect of the present disclosure, there is provided a mounting member including: a body portion provided in a camera head including an image sensor, the body portion being rotatable about a first axis that passes through the body portion and connectable with an endoscope; a locking protrusion provided in the body portion and configured to lock with the endoscope; a detachable button configured to be movable with respect to the body portion and control attachment and detachment of the endoscope to and from the body portion; and a spring whose load applied to the locking protrusion changes according to an advancing/retreating operation of the detachable button with respect to the body portion, wherein at least one of the body portion and the detachable button includes a through-hole forming a part of a flow path of a liquid to a space in which the locking protrusion in the body portion and the spring member are arranged.

### BRIEF DESCRIPTION OF THE DRAWINGS

FIG. 1 is a view illustrating a schematic configuration of an endoscopic device according to an embodiment;

FIG. 2 is a plan view illustrating a configuration of a main part of the endoscopic device illustrated in FIG. 1, and is a plan view illustrating configurations of a camera head body and a mounting member;

FIG. 3 is a view illustrating a connection portion between the camera head body and the mounting member;

FIG. 4 is an exploded perspective view (Part 1) of the mounting member according to the embodiment;

FIG. 5 is an exploded perspective view (Part 2) of the mounting member according to the embodiment;

FIG. 6 is a view illustrating a connection portion between a mounting member according to a first modification and the camera head body;

FIG. 7 is a plan view illustrating a configuration of a main part of an endoscopic device according to a second modification, and is a view illustrating a connection portion between a camera head body and a mounting member;

FIG. 8 is a plan view of the connection portion between the camera head body and the mounting member as viewed from a direction of an in-casing optical axis;

FIG. 9 is a plan view illustrating a configuration of a main part of an endoscopic device according to a third modification, and is a plan view illustrating configurations of a camera head body and a mounting member;

FIG. 10 is a view illustrating the connection portion between the camera head body and the mounting member;

FIG. 11 is a plan view of the mounting member as viewed from one direction of a first axis;

FIG. 12 is a plan view of the mounting member as viewed from the other direction of the first axis;

FIG. 13 is an exploded perspective view (Part 1) of the mounting member according to the third modification;

FIG. 14 is an exploded perspective view (Part 2) of the mounting member according to the third modification; and

FIG. 15 is an enlarged cross-sectional view of a configuration of a part of the camera head body.

### DETAILED DESCRIPTION

Hereinafter, modes for carrying out the present disclosure (hereinafter, referred to as “embodiments”) will be described in detail with reference to the drawings. Incidentally, the present disclosure is not limited by the following embodiments. In addition, the respective drawings referred to in the following description merely illustrate shapes, sizes, and positional relationships in a schematic manner to such an extent that contents of the present disclosure may be understood. That is, the present disclosure is not limited to only the shapes, sizes, and positional relationships illustrated in the respective drawings. Further, the same reference sign will be assigned to the same components in the description of the drawings.

Schematic Configuration of Endoscopic Device

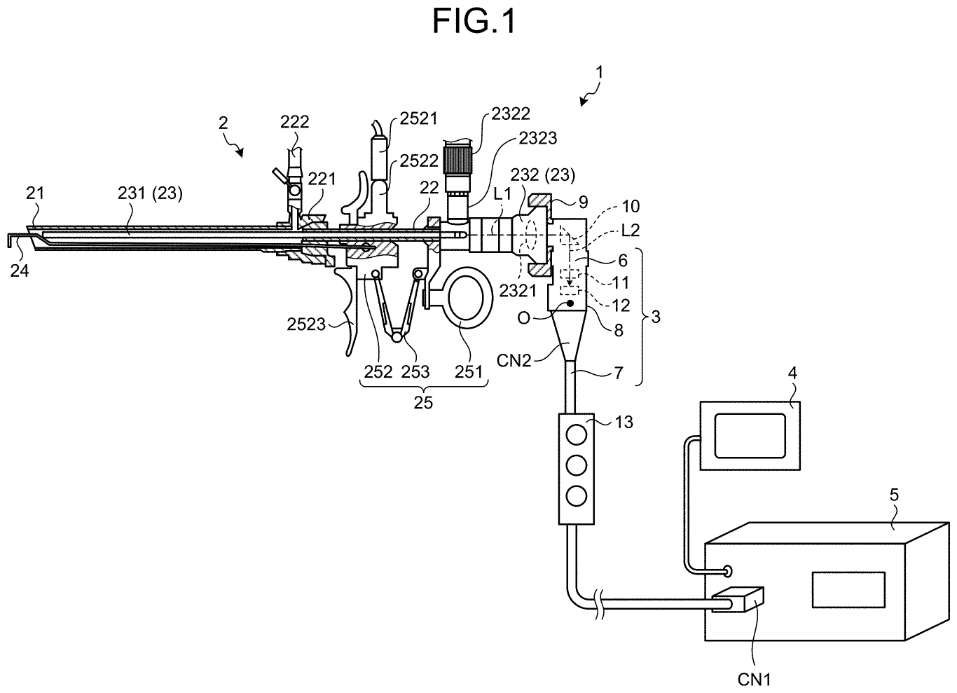

FIG. 1 is a view illustrating a schematic configuration of an endoscopic device according to an embodiment.

An endoscopic device 1 illustrated in FIG. 1 is a device used in the medical field to treat (for example, resect) a living tissue while observing the inside of a living body. As illustrated in FIG. 1, the endoscopic device 1 includes a resectoscope 2, an endoscope imaging device 3, a display device 4, and a control device 5.

Configuration of Resectoscope

The resectoscope 2 may be inserted into a living body, take a subject image, and treat a living tissue. As illustrated in FIG. 1, the resectoscope 2 includes a sheath 21, a guide tube 22, an endoscope 23, a resecto-electrode member 24, and a handle 25.

The sheath 21 has a cylindrical shape and is a portion to be inserted into a living body. The guide tube 22 has an outer diameter dimension smaller than an inner diameter dimension of the sheath 21, and is inserted into the sheath 21. A distal end side (left side in FIG. 1) of the guide tube 22 is fixed to the sheath 21 via an attachment member 221. Here, the attachment member 221 is provided with a water supply port 222 configured to inject a liquid into the sheath 21 and supply the liquid from a distal end of the sheath 21.

The endoscope 23 is a portion that takes a subject image, and includes an insertion unit 231 and an eyepiece unit 232 as illustrated in FIG. 1.

The insertion unit 231 is fixed in the guide tube 22 and inserted into the sheath 21. An optical system, which includes one or a plurality of lenses and condenses a subject image, is provided in the insertion unit 231.

The eyepiece unit 232 is connected to a proximal end (right end in FIG. 1) of the insertion unit 231. An eyepiece optical system 2321, which emits a subject image condensed by the optical system in the insertion unit 231 to the outside from the eyepiece unit 232, is provided in the eyepiece unit 232. The eyepiece unit 232 is formed in a tapered shape whose diameter increases toward the right side in FIG. 1, and the endoscope imaging device 3 is detachably connected to an enlarged diameter portion thereof.

Here, the eyepiece unit 232 is provided with a light source connector 2323 configured to connect a light guide 2322. That is, light supplied from a light source device (not illustrated) to the light guide 2322 is supplied to the insertion unit 231 via the eyepiece unit 232. The light supplied to the insertion unit 231 is emitted from a distal end of the insertion unit 231 and is emitted inside a living body. The light (subject image), which has been emitted inside the living body and reflected inside the living body, is emitted from the eyepiece unit 232 via the optical system in the insertion unit 231 and the eyepiece optical system 2321.

The resecto-electrode member 24 is inserted into the sheath 21 via the attachment member 221 and has a distal end protruding from the distal end of the sheath 21. A distal end portion of the resecto-electrode member 24 comes into contact with a living tissue, and treats the living tissue with a high-frequency current.

The handle 25 is a portion where a doctor or the like grips the resectoscope 2 and operates the resecto-electrode member 24. As illustrated in FIG. 1, the handle 25 includes a fixing ring 251, a slider 252, and a spring member 253.

The fixing ring 251 is a portion on which a doctor or the like hooks the thumb, and is fixed to the guide tube 22.

The slider 252 into which the guide tube 22 is inserted is movable in the left-right direction in FIG. 1 along the guide tube 22. As illustrated in FIG. 1, the resecto-electrode member 24 is fixed to the slider 252. That is, the resecto-electrode member 24 moves back and forth inside the sheath 21 in the left-right direction in FIG. 1 along with the movement of the slider 252. In addition, the slider 252 is provided with a power supply connector 2522 configured to connect a high frequency power cord 2521 connected to a high frequency power supply (not illustrated). The power supply connector 2522 is electrically connected to the resecto-electrode member 24 via a lead wire (not illustrated). Further, as illustrated in FIG. 1, the slider 252 is provided with a finger hook member 2523 configured for the doctor or the like to hook a finger other than the thumb and move the slider 252 (move the resecto-electrode member 24 back and forth).

The spring member 253 has a substantially U-shape and has one end attached to the fixing ring 251 and the other end attached to the slider 252. The spring member 253 biases the slider 252 to a side separating from the fixing ring 251. That is, the doctor or the like hooks the fingers on the fixing ring 251 and the finger hook member 2523, and pulls the finger hook member 2523 against a biasing force of the spring member 253, thereby moving the slider 252 to the right side in FIG. 1 (moving the resecto-electrode member 24 to the right side in FIG. 1). On the other hand, the slider 252 (resecto-electrode member 24) moves to the left side in FIG. 1 due to the biasing force of the spring member 253 when the doctor or the like releases the finger from the finger hook member 2523.

The endoscope imaging device 3 is detachably connected to the eyepiece unit 232 of the resectoscope 2 (endoscope 23). The endoscope imaging device 3 captures a subject image (subject image emitted from the eyepiece unit 232) taken by the endoscope 23 under the control of the control device 5, and outputs an image signal (RAW signal) obtained by this imaging. This image signal is, for example, an image signal of 4K or higher. The endoscope imaging device 3 includes an endoscope camera head 6 and a cable 7. Incidentally, a detailed configuration of the endoscope imaging device 3 will be described later.

The display device 4 is configured using a display using a liquid crystal or an organic electro luminescence (EL), or the like. The display device 4 displays an observation image based on a video signal from the control device 5 under the control of the control device 5.

The control device 5 is configured using a memory and a processor such as hardware of a field programmable gate array (FPGA) or a central processing unit (CPU). The control device 5 performs the overall control of operations of the endoscope imaging device 3, the display device 4, and the light source device (not illustrated). For example, the control device 5 performs predetermined image processing on the image signal (RAW signal) output from the endoscope imaging device 3 to generate a video signal for display, and displays an observation image based on this video signal on the display device 4.

Configuration of Endoscope Camera Head

Next, a detailed configuration of the endoscope camera head 6 will be described. As illustrated in FIG. 1, the endoscope camera head 6 and the cable 7 are provided.

The endoscope camera head 6 is a portion that is detachably connected to the eyepiece unit 232. As illustrated in FIG. 1, the endoscope camera head 6 includes a casing 8, a mounting member 9 (coupler), a prism 10, a lens unit 11, and an imaging unit 12.

The casing 8 is a casing that accommodates the prism 10, the lens unit 11, and the imaging unit 12 therein. In addition, the casing 8 has a lever 8a that switches a locked state with the eyepiece unit 232.

The mounting member 9 has a bottomed cylindrical shape into which the eyepiece unit 232 may be fitted. The mounting member 9 is regulated from moving in a direction of a first axis L1 with respect to the casing 8, and is rotatable with respect to the casing 8 about the first axis L1. Incidentally, a detailed configuration of the mounting member 9 will be described later.

The prism 10 is arranged on an in-casing optical axis L2 on the first axis L1, and deflects a traveling direction of the subject image taken by the endoscope 23. Specifically, the prism 10 deflects the traveling direction of the subject image emitted from the eyepiece unit 232 and taken in the casing 8 via an optical element (not illustrated) by approximately 90° to cause the subject image to travel along the in-casing optical axis L2.

The lens unit 11 is arranged on the in-casing optical axis L2. The lens unit 11 is configured using one or a plurality of lenses, and forms the subject image passing through the prism 10 on an imaging surface of the imaging unit 12. In addition, the lens unit 11 is provided with an optical zooming mechanism (not illustrated) that moves one or more lenses to change an angle of view under the control of the control device 5 or an operating unit 13, a focusing mechanism (not illustrated) that changes a focal point, and the like.

The imaging unit 12 is arranged on the in-casing optical axis L2. Then, the imaging unit 12 captures the subject image formed by the lens unit 11 under the control of the control device 5. The imaging unit 12 is configured using a sensor chip on which an imaging element (not illustrated), such as a charge coupled device (CCD) and a complementary metal oxide semiconductor (CMOS), which receives the subject image formed by the lens unit 11 and converts the subject image into an electrical signal, a signal processing unit (not illustrated) which performs signal processing (A/D conversion or the like) on the electrical signal (analog signal) from this imaging element to output an image signal, and the like are integrally formed, and outputs the image signal (RAW signal (digital signal)) after A/D conversion. Incidentally, the signal processing unit described above may be a separate body without being integrally formed with the imaging element.

In this manner, the endoscope camera head 6 is configured to be rotatable about the first axis L1 with respect to the eyepiece unit 232 of the endoscope 23 via the mounting member 9. In addition, the endoscope camera head 6 is configured so as to have a center of gravity O shifted from the first axis L1 (rotation center axis with respect to the eyepiece unit 232). Then, the endoscope camera head 6 is configured to rotate about the first axis L1 regardless of the rotation of the resectoscope 2 about the first axis L1 and take a posture in which the in-casing optical axis L2 set in the casing 8 extends along the vertical direction (posture in which the center of gravity O is located below the first axis L1).

The cable 7 has one end detachably connected to the control device 5 via a connector CN1, and the other end detachably connected to the endoscope camera head 6 via a connector CN2. The cable 7 transmits the image signal output from the endoscope camera head 6 to the control device 5, and also transmits each of a control signal, a synchronization signal, a clock, power, and the like output from the control device 5 to the endoscope camera head 6. Incidentally, in the transmission of the image signal from the endoscope camera head 6 to the control device 5 via the cable 7, the image signal may be transmitted as an optical signal or may be transmitted as an electrical signal. The same applies to the transmission of the control signal, the synchronization signal, and the clock from the control device 5 to the endoscope camera head 6 via the cable 7. In addition, as illustrated in FIG. 1, the cable 7 is provided with an operating unit 13 that receives instructions from the doctor or the like for various operations (for example, image quality adjustment (white balance adjustment, brightness adjustment, and the like) of the observation image) and an instruction to change the angle of view or the focal point of the lens unit 11).

Configuration of Mounting Member

Next, the detailed configuration of the mounting member 9 will be described.

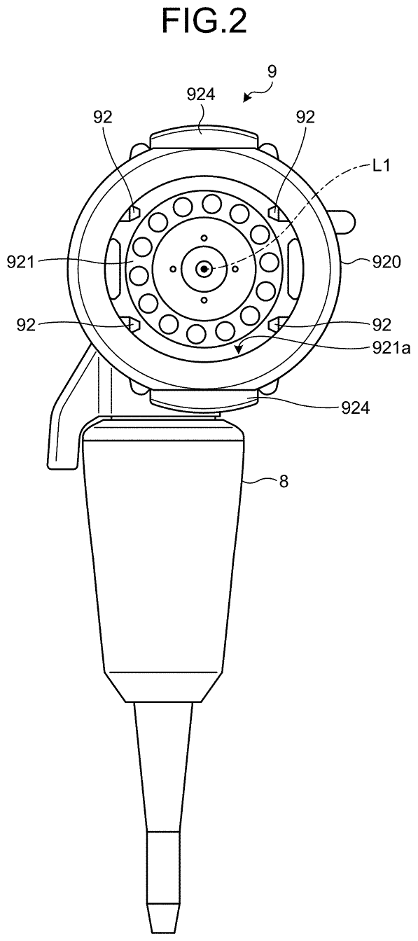

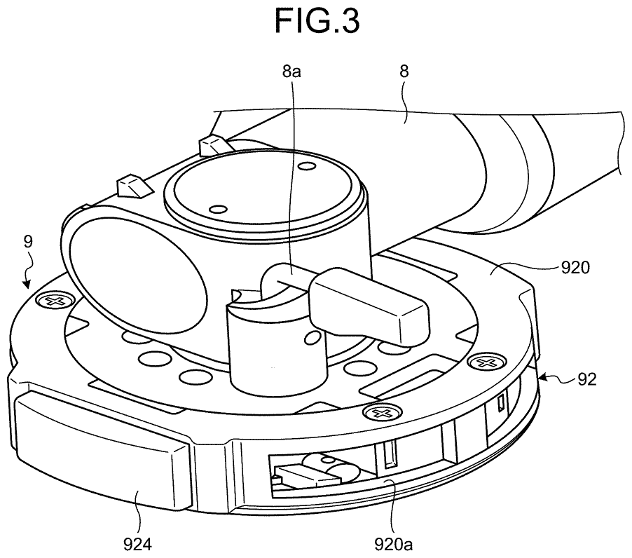

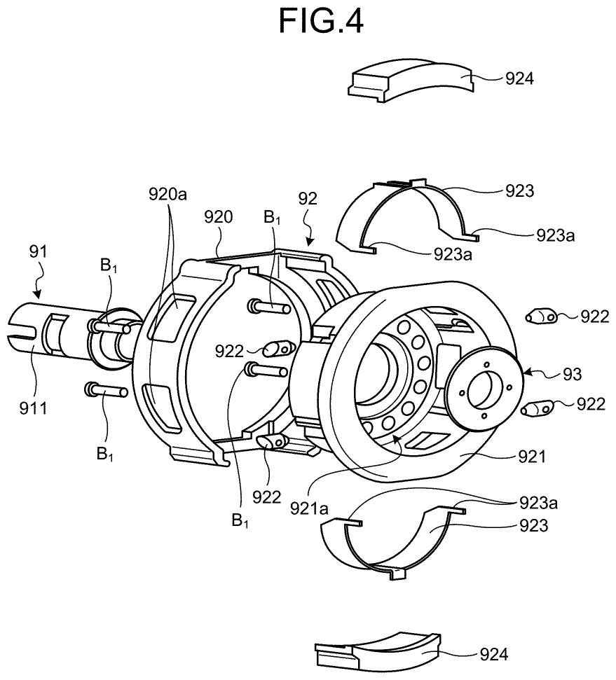

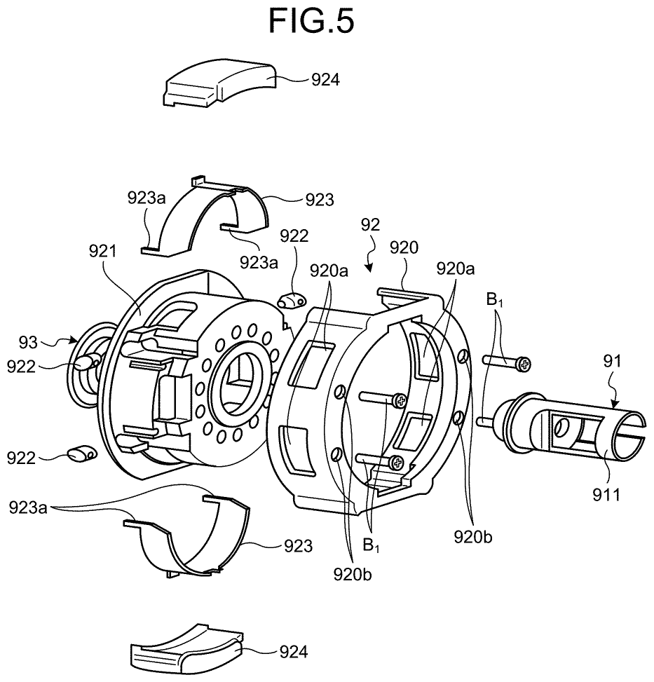

FIG. 2 is a plan view illustrating a configuration of a main part of the endoscopic device illustrated in FIG. 1, and is a plan view illustrating configurations of the camera head body and the mounting member. FIG. 3 is a view illustrating a connection portion between the camera head body and the mounting member. FIGS. 4 and 5 are exploded perspective views of the mounting member according to the embodiment. Incidentally, FIGS. 4 and 5 are exploded perspective views as viewed from different directions.

The mounting member 9 includes a first member 91, a second member 92, and a regulating member 93.

Configuration of First Member

First, a configuration of the first member 91 will be described.

The first member 91 is provided on the endoscope camera head 6 having the imaging unit 12. Specifically, the first member 91 is a member configured to attach the mounting member 9 to the casing 8 and a member configured to support the rotation of the second member 92. An end portion of the first member 91 on a side opposite to a side connected to the casing 8 is connected to the regulating member 93.

Configuration of Second Member

Next, a configuration of the second member 92 will be described.

The second member 92 may rotate about the first axis L1 passing through the first member 91, and the endoscope 23 may be connected to the second member 92. The second member 92 includes a first body portion 920, a second body portion 921, locking protrusions 922, spring members 923, and detachable buttons 924. In the mounting member 9, the body portion is constituted by the first body portion 920 and the second body portion 921.

The first body portion 920 and the second body portion 921 form a bottomed tubular casing.

The first body portion 920 has a ring shape and forms an outer peripheral side surface of the casing. A plurality of through-holes 920a that communicate the inside and the outside are formed on the outer peripheral side surface of the first body portion 920. In addition, the first body portion 920 is formed with a plurality of insertion holes 920b through which screws B₁ fixing the first body portion 920 and the second body portion 921 are inserted (see FIG. 5).

The second body portion 921 has a bottomed tubular shape. The eyepiece unit 232 is inserted into a concave portion 921a formed by the second body portion 921. In a state where the mounting member 9 is assembled, an outer peripheral surface of the second body portion 921 is covered with the first body portion 920 (see FIG. 3). Incidentally, the second body portion 921 is also formed with a hole in which the locking protrusion 922 protrudes and a screw hole (not illustrated) screwing with the screw B₁.

The locking protrusion 922 presses the endoscope 23 (eyepiece unit 232) when the endoscope 23 is connected. The mounting member 9 is provided with four locking protrusions 922. The locking protrusions 922 are arranged at positions where distal ends of the two locking protrusions 922 forming a pair face each other. The locking protrusion 922 is biased by the spring member 923 so as to be movable in directions of coming close to or separating from the paired locking protrusion 922, and abuts on an outer peripheral surface of the eyepiece unit 232 accommodated inside the mounting member 9 (in the concave portion 921a) and presses the eyepiece unit 232 toward a bottom of the concave portion 921a. The locking protrusion 922 is provided at a position to be rotated by 180° about the first axis L1 of the mounting member 9.

The spring member 923 is configured using a leaf spring formed by bending a strip-shaped member. The spring member 923 has both end portions 923a on which the locking protrusions 922 abut, and biases each of the locking protrusions 922 toward the paired locking protrusion 922.

The detachable button 924 is held by the first body portion 20 and the second body portion 921 so as to be capable of advancing and retreating with respect to the outer surface of the first body portion 920. At this time, the detachable button 924 is biased by the spring member 923 in a direction of projecting from the outer surface of the first body portion 920.

Configuration of Regulating Member

Next, a configuration of the regulating member 93 will be described.

The regulating member 93 regulates the movement of the second member 92 in the direction of the first axis L1 by rotatably sandwiching the second member 92 together with the first member 91.

In the mounting member 9 configured in this way, the locking protrusion 922 and the spring member 923 are accommodated in an internal space formed by the first body portion 920 and the second body portion 921.

When the detachable button 924 is pressed into the first body portion 920, the spring member 923 is deformed. Specifically, the detachable button 924 applies a load generated when the mounting member 9 is pressed toward the first axis L1 from the outside to the spring member 923. The spring member 923 expands when the load is applied. When the spring member 923 expands, a biasing force on the spring member 923 is reduced, and a biasing force on the locking protrusion 922 by the spring member 923 is reduced. When the biasing force on the locking protrusion 922 is reduced, the locking protrusion 922 may move in the direction of separating from the paired locking protrusion 922. In this state, the eyepiece unit 232 may be attached to the mounting member 9 or detached from the mounting member 9. When the pressing state of the detachable button 924 is released, the spring member 923 returns to the state before deformation. When the spring member 923 returns to the state before deformation, the locking protrusion 922 protrudes toward the paired locking protrusion 922 and is locked to the eyepiece unit 232. In this state, the eyepiece unit 232 is regulated from being detached from the mounting member 9.

In addition, the entire mounting member 9 is immersed in a cleaning liquid when cleaning the mounting member 9. At this time, the cleaning liquid or the like is introduced into an internal space where the locking protrusion 922 and the spring member 923 are arranged, the internal space being formed by the first body portion 920 and the second body portion 921, via the through-hole 920a formed in the first body portion 920. Further, the cleaning liquid or the like after cleaning is discharged through the through-hole 920a. Therefore, the through-hole 920a forms a part of a flow path of the cleaning liquid to the internal space formed by the first body portion 920 and the second body portion 921. Since the through-hole 920a is formed in the first body portion 920, the cleaning liquid may be introduced and discharged efficiently.

In the above-described embodiment, the through-hole 920a that communicates the internal space formed by the first body portion 920 and the second body portion 921 with the outside is formed in the first body portion 920 that forms a part of the casing of the mounting member 9. According to the present embodiment, the through-hole 920a promotes the infiltration of the cleaning liquid or the like into the internal space formed by the first body portion 920 and the second body portion 921 and the discharge, and thus, it is possible to improve the infiltration of the cleaning liquid into the inside of the mounting member 9 and the discharge to the outside. Since the introduction of the cleaning liquid into the mounting member 9 and the discharge are promoted, the cleanability of the mounting member 9 may be improved, and the time for cleaning may be shortened.

First Modification

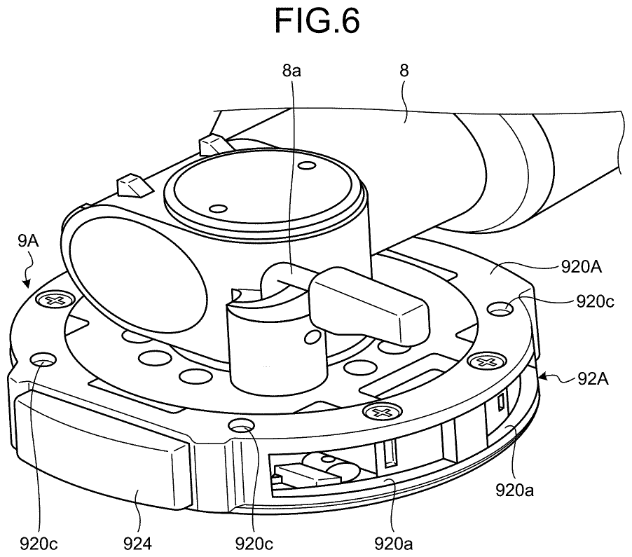

Subsequently, a first modification of the embodiment will be described. FIG. 6 is a view illustrating a connection portion between a mounting member according to the first modification and the camera head body. The first modification differs from the embodiment in terms that a mounting member 9A is provided in place of the mounting member 9 described above. Since configurations of the first modification other than the above-described configuration are the same as those of the endoscopic device 1 of the above-described embodiment, the description thereof will be omitted. The same reference signs will be assigned to the same components as those in the above-described embodiment.

The mounting member 9A includes the first member 91, a second member 92A, and the regulating member 93. Hereinafter, the second member 92A having a configuration different from that of the above-described embodiment will be described.

The second member 92A may rotate about the first axis L1 passing through the first member 91, and the endoscope 23 may be connected to the second member 92A. The second member 92A includes a first body portion 920A, the second body portion 921, the locking protrusions 922, the spring members 923, and the detachable buttons 924.

The first body portion 920A has a ring shape and forms an outer peripheral side surface of the casing. The first body portion 920A is formed with the plurality of through-holes 920a, the insertion holes 920b, and through-holes 920c that communicate an internal space formed by the first body portion 920 and the second body portion 921 with the outside. The through-hole 920c is formed on a surface different from a surface on which the through-hole 920a is formed, the surface being the same as a surface on which the insertion hole 920b is formed.

If the strength required for the first body portion 920A is satisfied, the infiltration and discharge of a cleaning liquid are promoted as the number of holes increases (the area opened by the holes increases).

Even in the first modification, the same effect as that of the above-described embodiment may be obtained.

Second Modification

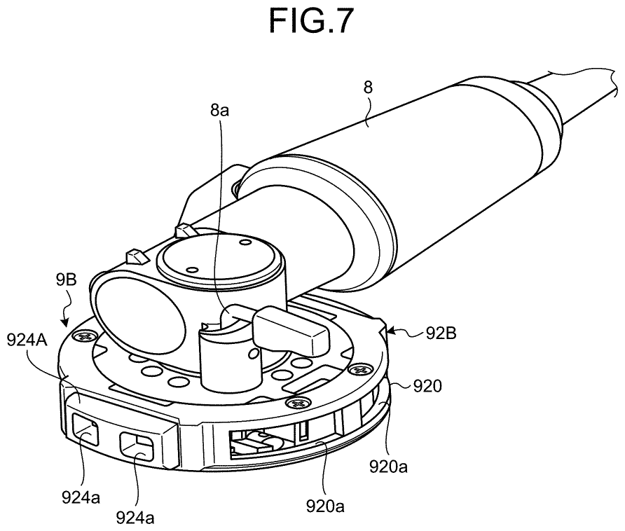

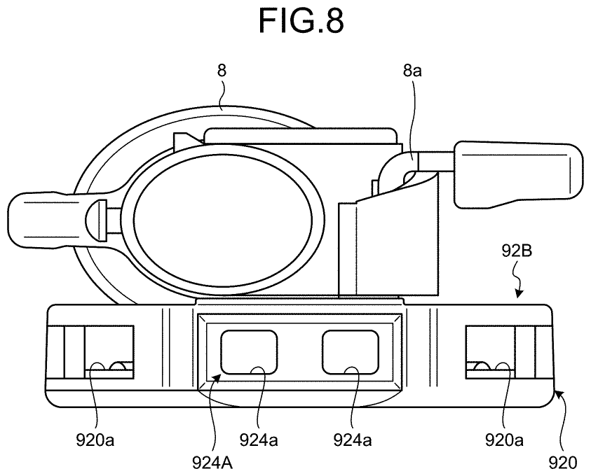

Subsequently, a second modification of the embodiment will be described. FIG. 7 is a plan view illustrating a configuration of a main part of an endoscopic device according to the second modification, and is a view illustrating a connection portion between a camera head body and a mounting member. FIG. 8 is a plan view of the connection portion between the camera head body and the mounting member as viewed from a direction of an in-casing optical axis. The second modification differs from the embodiment in terms that a mounting member 9B is provided in place of the mounting member 9 described above. Since configurations of the first modification other than the above-described configuration are the same as those of the endoscopic device 1 of the above-described embodiment, the description thereof will be omitted. The same reference signs will be assigned to the same components as those in the above-described embodiment.

The mounting member 9B includes the first member 91, a second member 92B, and the regulating member 93. Hereinafter, the second member 92B having a configuration different from that of the above-described embodiment will be described.

The second member 92B may rotate about the first axis L1 passing through the first member 91, and the endoscope 23 may be connected to the second member 92B. The second member 92B includes the first body portion 920, the second body portion 921, the locking protrusions 922, the spring members 923, and detachable buttons 924A.

The detachable button 924A applies a load from the outside to the spring member 923 to expand the spring member 923, and reduces a biasing force of the locking protrusion 922 caused by the spring member 923, which is similar to the detachable button 924. In addition, the detachable button 924A is formed with a plurality of through-holes 924a that communicates an internal space formed by the first body portion 920 and the second body portion 921 with the outside. The through-hole 924a penetrates in the advancing/retreating direction of the detachable button 924A, and forms an aperture smaller than a user's finger, for example. With such a size, it is possible to prevent the user's finger from being caught on the detachable button 924.

In this manner, the through-hole 924a may be formed in the detachable button 924A to promote the infiltration and discharge of a cleaning liquid. Incidentally, the second body portion 921 may be formed with a through-hole that is connected to the through-hole 924a and communicates with the concave portion 921a.

Even in the second modification, the same effect as that of the above-described embodiment may be obtained.

Third Modification

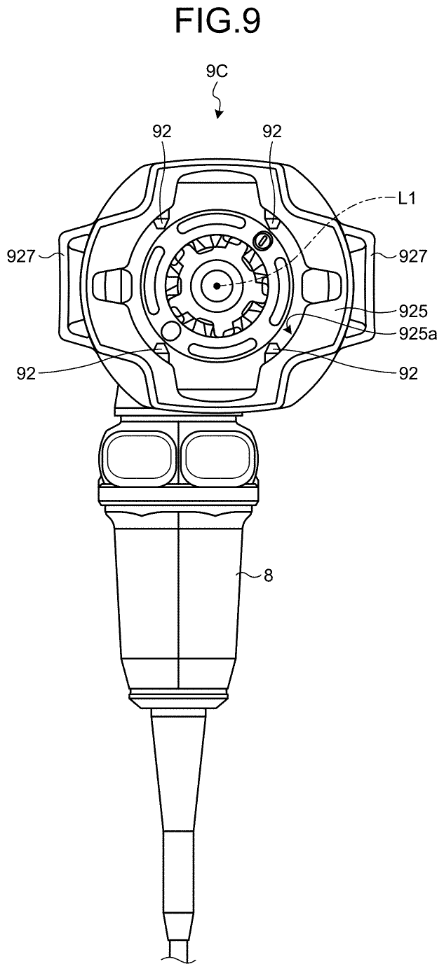

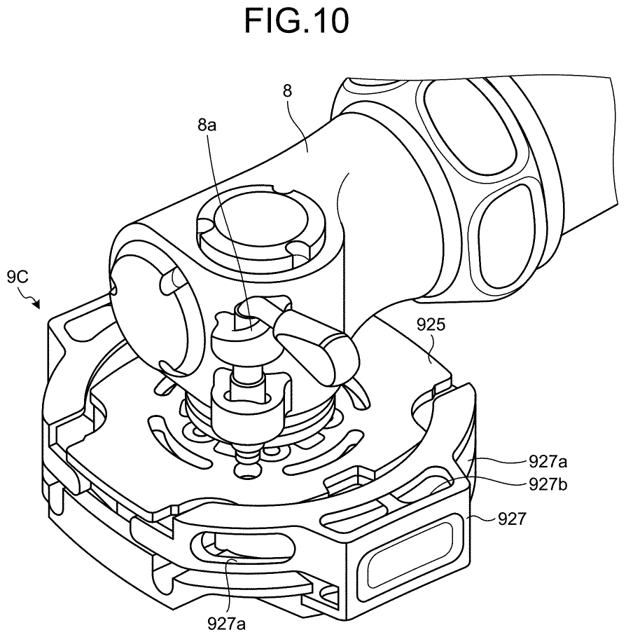

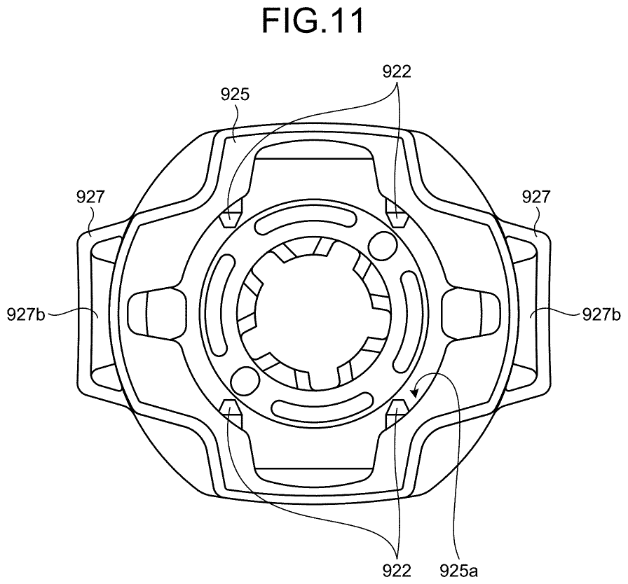

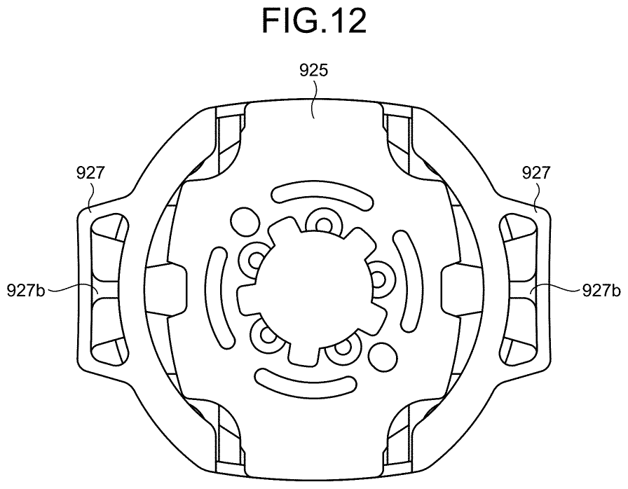

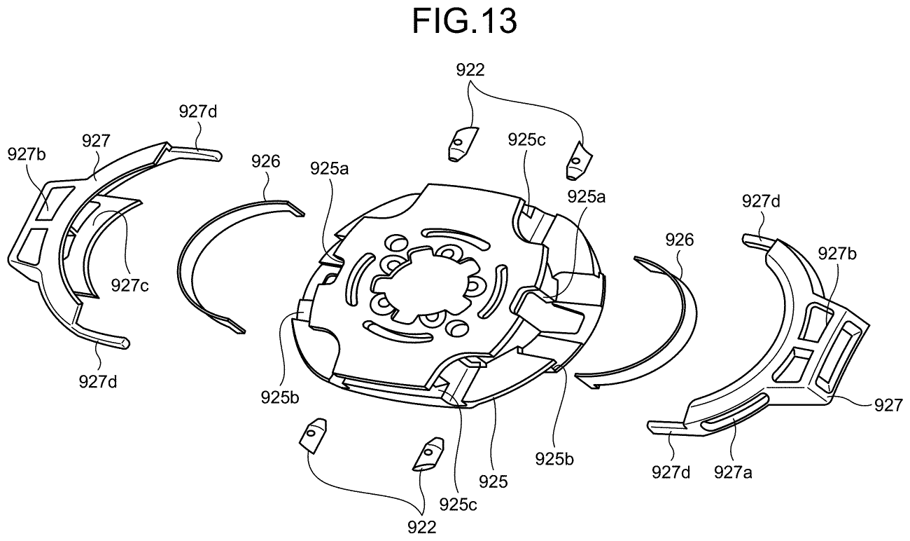

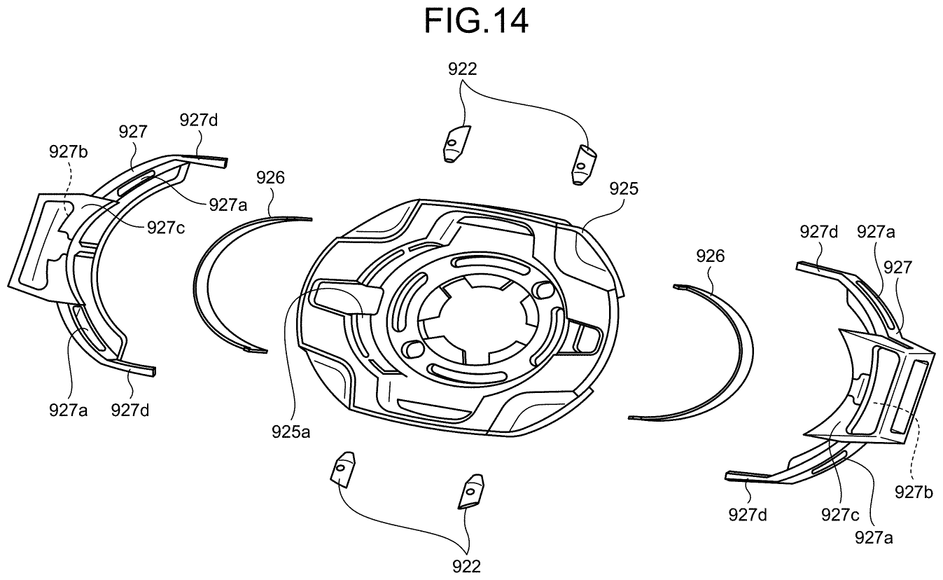

Subsequently, a third modification of the embodiment will be described. FIG. 9 is a plan view illustrating a configuration of a main part of an endoscopic device according to the third modification, and is a plan view illustrating configurations of a camera head body and a mounting member. FIG. 10 is a view illustrating a connection portion between the camera head body and the mounting member. FIG. 11 is a plan view of the mounting member as viewed from one direction of a first axis. FIG. 12 is a plan view of the mounting member as viewed from the other direction of the first axis. FIGS. 13 and 14 are exploded perspective views of the mounting member according to the third modification. Incidentally, FIG. 14 is an exploded perspective view as viewed from a direction different from that of FIG. 13.

The third modification differs from the embodiment in terms that a mounting member 9C is provided in place of the mounting member 9 described above. Since configurations of the third modification other than the above-described configuration are the same as those of the endoscopic device 1 of the above-described embodiment, the description thereof will be omitted. The same reference signs will be assigned to the same components as those in the above-described embodiment.

The mounting member 9C includes a first member, a second member 92C, and a regulating member. Hereinafter, the second member 92C having a configuration different from that of the above-described embodiment will be described. Incidentally, the same first member and the regulating member as the first member 91 and the regulating member 93 described above may be used, and shapes thereof may be changed in accordance with a shape of the second member 92C.

The second member 92C may rotate about the first axis L1 (see FIG. 1) passing through the first member 91, and the endoscope 23 may be connected to the second member 92C. The second member 92C includes a body portion 925, the locking protrusions 922, spring members 926, and detachable buttons 927. Incidentally, in the third modification, the locking protrusions 922 are arranged at positions where distal ends of the two locking protrusions 922 forming a pair face each other.

The body portion 925 forms a casing of the second member 92C. The eyepiece unit 232 is inserted into a concave portion 925a formed by the body portion 925.

The body portion 925 has a dish shape and includes a first guide portion 925b and a second guide portion 925c that guide a moving direction of the detachable button 927. Incidentally, the body portion 925 is also formed with a hole in which the locking protrusion 922 protrudes and a screw hole (not illustrated) screwing with the screw B₁.

Each of the first guide portion 925b and the second guide portion 925c has a concave shape extending along the advancing/retreating direction of the detachable button 927.

The spring member 926 is configured using a leaf spring formed by bending a strip-shaped member. The spring member 926 has both end portions on which the locking protrusions 922 abut, and biases each of the locking protrusions 922 toward the paired locking protrusion 922.

The detachable button 927 has an arc shape, applies a load from the outside to the spring member 926 to expand the spring member 926, and reduces a biasing force on the locking protrusion 922 caused by the spring member 926. In addition, the detachable button 927 is formed with through-holes 927a and 927b that communicate the external side with the body portion 925 side. The through-hole 927a is provided between an end portion and a central portion of the detachable button 927, and penetrates the outside and the inside. The through-hole 927b is provided in the central portion of the detachable button 927, and has an L-shaped hole that extends in the advancing/retreating direction of the detachable button 927 after extending in a direction orthogonal to the advancing/retreating direction of the detachable button 927 with respect to the body portion 925. Incidentally, either the through-hole 927a or 927b may be formed.

In addition, the detachable button 927 includes a first sliding portion 927c that extends in the advancing/retreating direction to the body portion 925 and slides along the first guide portion 925b, and a second sliding portion 927d that moves along the second guide portion 925c. The first sliding portion 927c has a plate shape extending with a width equivalent to a notch width of the first guide portion 925b. The second sliding portion 927d extends in a columnar shape that is movable along a recess of the second guide portion 925c.

In the mounting member 9C, the spring member 926 is arranged between the body portion 925 and the detachable button 927. As one of the body portion 925 and the detachable button 927 is locked with the other, the detachable button 927 is prevented from being detached from the body portion 925. For example, the detachable button 927 is provided with a protrusion that locks with the body portion 925 to prevent the detachable button 927 from being detached from the body portion 925. At this time, the locking protrusion 922 protrudes toward the paired locking protrusion 922.

When the detachable button 927 is pressed toward the body portion 925, the spring member 926 is deformed. When the spring member 926 is deformed, the locking protrusion 922 may move in a direction of separating from the paired locking protrusion 922. In this state, the eyepiece unit 232 may be attached to the mounting member 9C or detached from the mounting member 9C. When the pressing state of the detachable button 927 is released, the spring member 926 returns to the state before deformation. When the spring member 926 returns to the state before deformation, the locking protrusion 922 protrudes toward the paired locking protrusion 922 and is locked to the eyepiece unit 232. In this state, the eyepiece unit 232 is regulated from being detached from the mounting member 9C.

In addition, when cleaning the mounting member 9C, a cleaning liquid or the like is introduced into a gap formed by the detachable button 927 and the body portion 925 and the like via the through-holes 927a and 927b formed in the detachable button 927. Further, the cleaning liquid or the like after cleaning is discharged through the through-hole 927a. Since the through-holes 927a and 927b are formed in the detachable button 927 in this manner, the cleaning liquid may be introduced and discharged efficiently.

Even in the third modification, the same effect as that of the above-described embodiment may be obtained.

Incidentally, the body portion 925 may be formed with a hole penetrating the body portion 925 in the third modification. For example, a through-hole may be formed in the second guide portion 925c. When the hole is formed in the body portion 925, the cleaning liquid may be evenly distributed throughout the entire body portion 925.

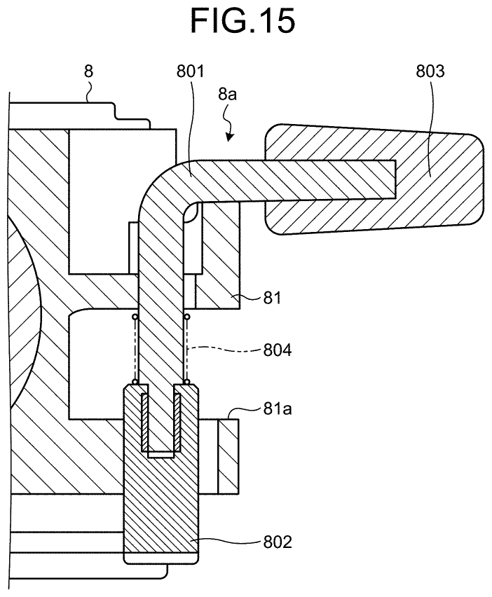

In addition, the casing 8 exposes and holds a part of the lever 8a in order to improve the cleanability of the lever 8a in the third modification. FIG. 15 is an enlarged cross-sectional view of a configuration of a part of the camera head body. As illustrated in FIG. 15, the lever 8a includes: a shaft portion 801 that bends in an L shape; an insertion/removal portion 802 provided at one end of the shaft portion 801, held by the casing 8, and inserted into or removed from a hole formed in the mounting member 9C (body portion 925) by the rotation of the lever 8a; and an operating unit 803 provided at the other end of the shaft portion 801 and operated by a user. In addition, in the casing 8, a holding portion 81 that holds the lever 8a is notched, and a notched portion 81a that exposes a part of the lever 8a is formed. A spring 804 is provided in a portion of the lever 8a where the notched portion 81a is formed. The spring 804 orbits the shaft portion 801 and biases the insertion/removal portion 802. When the shaft portion 801 of the lever 8a is rotated by the operation of the operating unit 803, the insertion/removal portion 802 protrudes from the holding portion 81 due to the biasing of the spring 804. In FIG. 15, the insertion/removal portion 802 moves downward. The insertion/removal portion 802 protruding from the holding portion 81 is inserted into a hole (not illustrated) formed in the body portion 925 to regulate the rotation of the body portion 925. When the lever 8a returns to the original position, the insertion/removal portion 802 is separated from the hole of the body portion 925 again, and the body portion 925 becomes rotatable.

In the configuration illustrated in FIG. 15, a cleaning liquid may be distributed inside the holding portion 81 by the notched portion 81a when cleaning the endoscope camera head 6. Specifically, the shaft portion 801, the insertion/removal portion 802, and the spring 804 may be immersed in the cleaning liquid through the notched portion 81a. At this time, the lever 8a may be cleaned more effectively by operating and rotating the lever 8a.

### Other Embodiments

Variations may be formed by appropriately combining a plurality of components disclosed in the endoscopic device according to the above-described embodiment of the present disclosure. For example, some components may be deleted from all the components described in the endoscopic device according to the above-described embodiment of the present disclosure. Further, the components described in the endoscopic devices according to the above-described embodiment and modifications of the present disclosure may be appropriately combined.

Although some of the embodiments of the present application have been described in detail with reference to the drawings as above, these are merely examples, and it is possible to carry out the present disclosure in other forms in which various modifications and improvements have been made based on the knowledge of those skilled in the art, including the aspects described in the disclosure.

Incidentally, the present technique may also adopt the following configurations.

(1) A mounting member including:

a body portion provided in a camera head including an image sensor, the body portion being rotatable about a first axis that passes through the body portion and connectable with an endoscope;

a locking protrusion provided in the body portion and configured to lock with the endoscope;

a detachable button configured to be movable with respect to the body portion and control attachment and detachment of the endoscope to and from the body portion; and

a spring whose load applied to the locking protrusion changes according to an advancing/retreating operation of the detachable button with respect to the body portion, wherein

at least one of the body portion and the detachable button includes a through-hole forming a part of a flow path of a liquid to a space in which the locking protrusion in the body portion and the spring member are arranged.

(2) The mounting member according to (1), wherein the body portion includes:

an annular first member configured to form an outer peripheral side surface of the body portion; and

a second member including a concave portion configured to accommodate the endoscope, the second member being accommodated in the first member, and

the through-hole is formed in the first member and configured to penetrate an outer circumference and an inner circumference of the first member.

(3) The mounting member according to (2), wherein the through-hole is formed on the outer peripheral side surface of the body portion.
(4) The mounting member according to any one of (1) to (3), wherein the through-hole is formed in the detachable button and penetrates in an advancing/retreating direction with respect to the body portion.
(5) The mounting member according to (1), wherein

the body portion has a dish shape including a concave portion configured to accommodate the endoscope,

the detachable button has an arc shape extending along an outer circumference of the body portion, and

the through-hole is formed in the detachable button and penetrates in an advancing/retreating direction with respect to the body portion.

(6) The mounting member according to (5), wherein

the detachable button includes a sliding portion extending in the advancing/retreating direction with respect to the body portion and configured to slide with respect to the body portion, and

the body portion includes a guide portion configured to guide a moving direction of the sliding portion.

(7) An endoscopic device including:

the mounting member according to (1); and

an endoscope configured to capture and emit a subject image.

As described above, the mounting member and the endoscopic device according to the present disclosure are advantageous in terms of improving the infiltration of the cleaning liquid into the inside of the member and the discharge to the outside.

According to the present disclosure, it is possible to improve the infiltration of a cleaning liquid into the inside of a member and the discharge of the cleaning liquid to the outside.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A mounting member comprising:
a body portion provided in a camera head including an image sensor, the body portion being rotatable about a first axis that passes through the body portion and connectable with an endoscope;
a locking protrusion provided in the body portion and configured to lock with the endoscope;
a detachable button configured to be movable with respect to the body portion and control attachment and detachment of the endoscope to and from the body portion; and
a spring whose load applied to the locking protrusion changes according to an advancing/retreating operation of the detachable button with respect to the body portion, wherein
at least one of the body portion and the detachable button includes a through-hole forming a part of a flow path of a liquid to a space in which the locking protrusion in the body portion and the spring member are arranged.

2. The mounting member according to claim 1, wherein the body portion includes:
an annular first member configured to form an outer peripheral side surface of the body portion; and
a second member including a concave portion configured to accommodate the endoscope, the second member being accommodated in the first member, and
the through-hole is formed in the first member and configured to penetrate an outer circumference and an inner circumference of the first member.

3. The mounting member according to claim 2, wherein the through-hole is formed on the outer peripheral side surface of the body portion.

4. The mounting member according to claim 1, wherein the through-hole is formed in the detachable button and penetrates in an advancing/retreating direction with respect to the body portion.

5. The mounting member according to claim 1, wherein
the body portion has a dish shape including a concave portion configured to accommodate the endoscope,
the detachable button has an arc shape extending along an outer circumference of the body portion, and
the through-hole is formed in the detachable button and penetrates in an advancing/retreating direction with respect to the body portion.

6. The mounting member according to claim 5, wherein
the detachable button includes a sliding portion extending in the advancing/retreating direction with respect to the body portion and configured to slide with respect to the body portion, and
the body portion includes a guide portion configured to guide a moving direction of the sliding portion.

7. An endoscopic device comprising:
the mounting member according to claim 1; and
an endoscope configured to capture and emit a subject image.
